(19) United States
(12) Patent Application Publication
(10) Pub. No.: US 2025/0353989 A1
(43) Pub. Date: Nov. 20, 2025

(54) METHOD FOR DEGRADING POLYCARBONATE

(71) Applicant: NAN YA PLASTICS CORPORATION, TAIPEI (TW)

(72) Inventors: CHING-YAO YUAN, TAIPEI (TW); WEI-SHENG CHENG, TAIPEI (TW); CHAO-TUNG WU, TAIPEI (TW)

(21) Appl. No.: 18/764,164

(22) Filed: Jul. 4, 2024

(30) Foreign Application Priority Data
May 16, 2024 (TW) 113118051

(51) Int. Cl. C08J 11/24; C07C 37/055; C07C 39/16; C08G 64/06; C08J 11/14; C08J 11/16; C08L 69/00

(52) U.S. Cl. CPC C08J 11/24; C07C 37/0555; C07C 39/16; C08G 64/06; C08J 11/14; C08J 11/16; C08L 69/00; C08J 2369/00; C08L 2207/20

(57) ABSTRACT

A method for degrading polycarbonate includes providing a depolymerizing solvent, in which the depolymerizing solvent is phenol; adding a metal hydroxide to the depolymerizing solvent to form a mixed liquid; and adding a polycarbonate material to the mixed liquid and adding a predetermined amount of water to the mixed liquid to form a reaction liquid. An added concentration of the metal hydroxide is not less than 100 ppm, and the water content in the reaction liquid is controlled to be not greater than 10 wt %. The polycarbonate material undergoes a depolymerization reaction to form bisphenol A and carbon dioxide.

### CROSS-REFERENCE TO RELATED PATENT APPLICATION

This application claims the benefit of priority to Taiwan Patent Application No. 113118051, filed on May 16, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a chemical method, and more particularly to a method for degrading polycarbonate.

### BACKGROUND OF THE DISCLOSURE

In the related art, US 2023/0382837 A1 (Applicant: LG) and WO 2020/257234 A1 (Applicant: Sabic) disclose methods that use low carbon alcohols (e.g., methanol or ethanol) and cosolvents (e.g., toluene or dichloromethane) for depolymerizing polycarbonate. The use of cosolvents can enhance the depolymerization efficiency. However, the cosolvents used in the related art may remain in the BPA product, thereby affecting subsequent reactions, and the toxic cosolvents may increase the difficulty of subsequent processing.

### SUMMARY OF THE DISCLOSURE

In response to the above-referenced technical inadequacies, the present disclosure provides a method for degrading polycarbonate.

In one aspect, the present disclosure provides a method for degrading polycarbonate, which includes a preparation operation, an addition operation, and a depolymerization operation. The preparation operation includes providing a depolymerizing solvent, in which the depolymerizing solvent is phenol. The addition operation includes adding a metal hydroxide to the depolymerizing solvent to form a mixed liquid. The depolymerization operation includes adding a polycarbonate material to the mixed liquid, and adding a predetermined amount of water to the mixed liquid to form a reaction liquid. Further, an added concentration of the metal hydroxide is not less than 100 ppm, and a water content in the reaction liquid is controlled to be not greater than 10 wt %. The polycarbonate material undergoes a depolymerization reaction in the depolymerization operation to form bisphenol A (BPA) and carbon dioxide (CO₂).

In certain embodiments, the preparation operation further includes heating the depolymerizing solvent to a first heating temperature ranging from 60° C. to 100° C. The depolymerization operation further includes heating the reaction liquid to a second heating temperature ranging from 110° C. to 150° C.

In certain embodiments, the first heating temperature ranges from 70° C. to 90° C., and the second heating temperature ranges from 110° C. to 140° C.

In certain embodiments, the depolymerization operation further includes controlling the water content in the reaction liquid to be between 0.5 wt % and 10 wt % during the depolymerization reaction.

In certain embodiments, when the water content in the reaction liquid is consumed to be below 0.5 wt %, the depolymerization operation further includes replenishing water to the reaction liquid to adjust the water content in the reaction liquid to be between 0.5 wt % and 10 wt %.

In certain embodiments, an initial weight ratio of the depolymerizing solvent relative to the polycarbonate material in the reaction liquid ranges between 1 to 14.

In certain embodiments, an intermediate product formed by the depolymerization of the polycarbonate material is diphenyl carbonate, which is further depolymerized into phenol and carbon dioxide (CO₂) during the depolymerization reaction; and a weight ratio of the depolymerizing solvent relative to the polycarbonate material continuously increases during the depolymerization reaction.

In certain embodiments, the metal hydroxide is at least one material selected from the group consisting of alkali metal (Group 1A metal) hydroxides, alkaline earth metal (Group 2A metal) hydroxides, and transition metal hydroxides.

In certain embodiments, the metal hydroxide is at least one of sodium hydroxide (NaOH) and potassium hydroxide (KOH).

In certain embodiments, the added concentration of the metal hydroxide ranges from 500 ppm to 10,000 ppm.

In certain embodiments, the addition operation includes adding an aqueous solution containing the metal hydroxide to the depolymerizing solvent to form the mixed liquid.

In certain embodiments, a weight percentage concentration of the metal hydroxide in the aqueous solution ranges from 15 wt % to 60 wt %.

Therefore, in the method for degrading polycarbonate by the present disclosure, by virtue of “performing a preparation operation, which includes: providing a depolymerizing solvent, in which the depolymerizing solvent is phenol (phenol); performing an addition operation, which includes: adding a metal hydroxide to the depolymerizing solvent to form a mixed liquid; and performing a depolymerization operation, which includes: adding a polycarbonate (PC) material to the mixed liquid and adding a predetermined amount of water to the mixed liquid to form a reaction liquid,” and “an added concentration of the metal hydroxide being not less than 100 ppm, and a water content in the reaction liquid being controlled to be not greater 10 wt %,” the chemical reaction can incline towards a de-polymerization reaction, thereby increasing the de-polymerization conversion rate of polycarbonate and the yield of bisphenol A (BPA).

The method for degrading polycarbonate provided by the present disclosure effectively avoids the problem of cosolvent residues (e.g., toluene or dichloromethane) in the product (e.g., BPA) as mentioned in the related art.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic flow diagram of a method for degrading polycarbonate according to an embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### Method for Degrading Polycarbonate

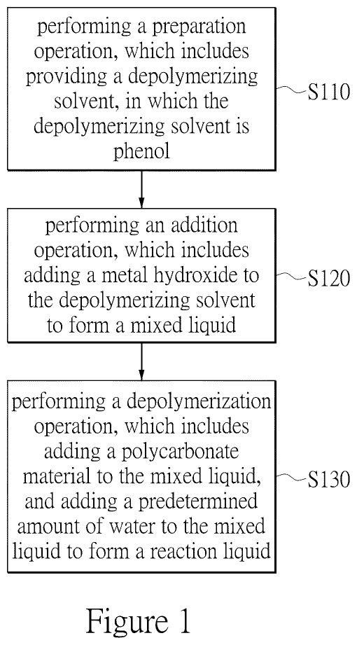

Referring to FIG. 1, an embodiment of the present disclosure aims to provide a method for degrading polycarbonate (PC), particularly related to a transesterification method for degrading polycarbonate. The method for degrading polycarbonate according to the embodiment of the present disclosure effectively avoids the issue of cosolvent residues (e.g., toluene or dichloromethane) being left in the product (BPA) as mentioned in the related art.

Specifically, the method for degrading polycarbonate (PC) according to the embodiment of the present disclosure includes step S110, step S120, and step S130. It should be noted that the sequence of steps and the actual operation methods described in the present embodiment can be adjusted according to requirements and are not limited to those described in the present embodiment.

Step S110 is to perform a preparation operation, which includes: providing a depolymerizing solvent and adding the depolymerizing solvent to a reaction tank. The depolymerizing solvent is phenol. More specifically, the preparation operation in the present embodiment adopts phenol as the single (sole/unitary) depolymerizing solvent.

In the present embodiment, the preparation operation further includes heating the depolymerizing solvent up to a first heating temperature.

The first heating temperature ranges from 60° C. to 100° C., preferably ranges from 70° C. to 90° C., and more preferably ranges from 75° C. to 85° C. For example, the first heating temperature is 80° C., but the present disclosure is not limited thereto.

Step S120 is to perform an addition operation, which includes adding a metal hydroxide to the depolymerizing solvent in the reaction tank to form a mixed liquid.

The metal hydroxide is composed of hydroxide (OH⁻) anions and metal cations Mn⁺, and the metal hydroxide dissociates in the presence of water.

In some embodiments of the present disclosure, the metal hydroxide is at least one material selected from the group consisting of alkali metal (Group 1A metal) hydroxides, alkaline earth metal (Group 2A metal) hydroxides, and transition metal hydroxides.

For example, the alkali metal (Group 1A metal) hydroxides can be sodium hydroxide (NaOH) or potassium hydroxide (KOH). The alkaline earth metal (Group 2A metal) hydroxides can be magnesium hydroxide (Mg(OH)₂) or calcium hydroxide (Ca(OH)₂). Additionally, the transition metal hydroxides can be manganese hydroxide (Mn(OH)₂), but the present disclosure is not limited thereto. In the present embodiment, the metal hydroxide is preferably at least one of sodium hydroxide (NaOH) and potassium hydroxide (KOH).

Furthermore, in the mixed liquid, an addition concentration of the metal hydroxide is not less than 100 ppm (parts per million), preferably between 100 ppm and 200,000 ppm, and more preferably between 500 ppm and 10,000 ppm. Specifically, the addition concentration of the metal hydroxide is between 829 ppm and 8,719 ppm, but the present disclosure is not limited thereto.

Moreover, the addition operation of the embodiment of the present disclosure involves adding an aqueous solution containing the metal hydroxide into the depolymerizing solvent to be mixed with each other, thereby forming the mixed liquid containing the depolymerizing solvent, metal hydroxide, and water. The metal hydroxide dissociates in the presence of water into hydroxide anions (OH⁻) and metal cations Mn⁺ (e.g., Na⁺ or K⁺), which can catalyze the depolymerization of the subsequently added polycarbonate (PC).

In the aqueous solution, a weight percentage concentration of the metal hydroxide is between 15 wt % and 60 wt %, preferably between 20 wt % and 45 wt %, and more preferably between 25 wt % and 40 wt %. For example, in one embodiment of the present disclosure, the metal hydroxide added in the aqueous solution is sodium hydroxide (NaOH) by a weight percentage concentration of 32 wt %, but the present disclosure is not limited thereto.

Additionally, an added amount of the aqueous solution (containing the metal hydroxide) added into the depolymerizing solvent is approximately between one-four hundredth ( 1/400) and one-twentieth ( 1/20) of the depolymerizing solvent, and the concentration of the metal hydroxide in the depolymerizing solvent can be adjusted within the aforementioned concentration range (e.g., not less than 100 ppm, preferably between 100 and 200,000 ppm, and more preferably between 500 and 100,000 ppm) through the added amount of the aqueous solution. However, the present disclosure is not limited to the aforementioned embodiment. The addition operation of the present disclosure can also involve directly adding powders of the metal hydroxide to the depolymerizing solvent, followed by adding an appropriate amount of water to form the mixed liquid containing a specific concentration of the metal hydroxide.

Step S130 is to perform a depolymerization operation, which includes: adding a polycarbonate material into the mixed liquid in the reaction tank and selectively adding a predetermined amount of water to the mixed liquid to form a reaction liquid.

Accordingly, the reaction liquid contains water, depolymerizing solvent, metal hydroxide, and polycarbonate material, in which the water content in the reaction liquid is controlled to be not greater than 10 wt %.

In the present embodiment, the polycarbonate material is polycarbonate particles to be depolymerized, which can be obtained from crushing recycled polycarbonate waste, but the present disclosure is not limited thereto.

The depolymerization operation further includes heating the reaction liquid up to a second heating temperature, so that the polycarbonate material undergoes a depolymerization reaction, ultimately forming bisphenol A (BPA) product and carbon dioxide (CO₂) gas (by product).

More specifically, the depolymerization operation involves heating the reaction liquid from the first temperature (e.g., 60° C. to 100° C.) to the second heating temperature, and the second heating temperature ranges from 110° C. to 150° C., preferably ranges from 110° C. to 140° C., and more preferably ranges from 110° C. to 135° C. For example, the second heating temperature is 120° C., but the present disclosure is not limited thereto.

Furthermore, the depolymerization operation involves continuously stirring the reaction liquid for 1 hour to 10 hours, preferably 2 hours to 8 hours, and more preferably 3 hours to 6 hours after heating the reaction liquid to the second heating temperature to ensure that the depolymerization reaction is carried out fully.

It is worth mentioning that, in the present embodiment, the depolymerization operation further includes: controlling the water content in the reaction liquid to be between 0.5 wt % and 10 wt %, preferably between 1 wt % and 10 wt %, and more preferably between 1.4 wt % and 9 wt % during the depolymerization reaction.

Accordingly, the polycarbonate material can achieve a high depolymerization conversion rate under the conditions of the added concentration of the metal hydroxide (e.g., not less than 100 ppm) and the water content (e.g., 0.5 to 10 wt %).

In one embodiment of the present disclosure, an initial weight proportion (i.e., feed weight proportion) of the depolymerizing solvent relative to the polycarbonate material in the reaction liquid is between 300 to 700:50 to 300, preferably between 400 to 600:100 to 300, and more preferably between 450 to 550:150 to 250.

In other words, an initial weight ratio of the depolymerizing solvent relative to the polycarbonate material in the reaction liquid (i.e., the ratio value of the initial amount of the depolymerizing solvent divided by the initial amount of the polycarbonate material) ranges between 1 to 14, preferably between 1.5 to 5.0, and more preferably between 2 to 3.

For example, an initial amount of the depolymerizing solvent (phenol) is 500 parts by weight, and the initial amount of the polycarbonate material (PC) is 200 parts by weight. Therefore, the initial weight ratio of the depolymerizing solvent to the polycarbonate material is 2.5 (i.e., 500/200).

Furthermore, in the depolymerization operation (i.e., step S130), the water content in the reaction liquid is controlled to be between 0.5 wt % and 10 wt %. The control method can involve, for example, taking out a small amount of the reaction liquid from the reaction tank (e.g., 1 ml to 10 ml of reaction liquid) and using a water content meter to measure the water content in the reaction liquid.

It is worth mentioning that since the depolymerization reaction consumes water, the depolymerization reaction reduces the water content in the reaction liquid. The method of the embodiment of the present disclosure monitors the water content in the reaction liquid during the depolymerization reaction. When the water content in the reaction liquid is consumed to be below 0.5 wt %, the depolymerization operation can further include replenishing water to the reaction liquid to adjust the water content in the reaction liquid to be between 0.5 wt % and 10 wt %. Accordingly, the afore-mentioned operation method helps further depolymerize the polycarbonate material without decomposing the bisphenol A (BPA) product, thereby improving the yield of the bisphenol A product.

Additionally, it is worth mentioning that an intermediate formed by the depolymerization of the polycarbonate material is diphenyl carbonate (DC), which will further be depolymerized into phenol (R—OH) and carbon dioxide (CO₂) during the depolymerization reaction.

Overall, the depolymerization reaction follows the sequence of chemical reaction mechanism 1 and chemical reaction mechanism 2 described below.

Chemical Reaction Mechanism 1: The polycarbonate (PC) material is firstly depolymerized into bisphenol A (BPA) product and diphenyl carbonate (DC) intermediate in the presence of the depolymerizing solvent (i.e., phenol, R—OH, where R is a phenyl group), the metal hydroxide (i.e., catalyst, M-OH, where M is a metal), and water (i.e., H₂O).

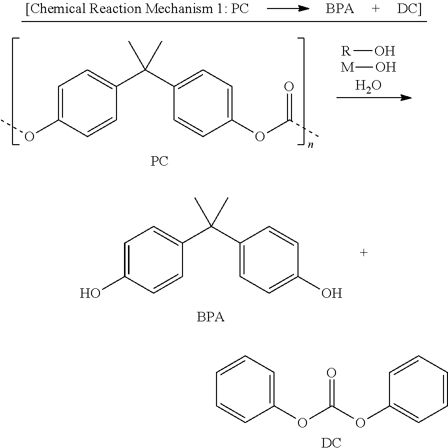

Chemical Reaction Mechanism 2: The diphenyl carbonate (DC) intermediate produced in the depolymerization reaction further depolymerizes into phenol (R—OH) and carbon dioxide (CO₂) gas in the presence of water.

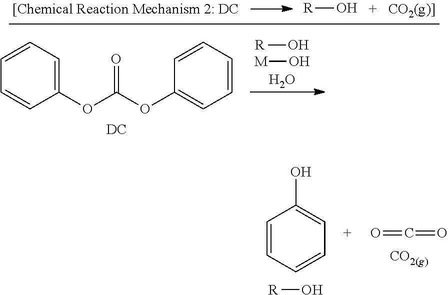

In Chemical Reaction Mechanism 1, the bisphenol A (BPA) product generated under the afore-mentioned conditions (i.e., the water content being controlled to be between 0.5 wt % and 10 wt %) does not undergo further decomposition, and only the diphenyl carbonate intermediate DC decomposes, thus preserving the bisphenol A product and increasing the yield of the bisphenol A product.

Therefore, under the above-mentioned water content and metal oxide concentration, the polycarbonate material ultimately forms bisphenol A (BPA) as the main product and phenol (R—OH) as a byproduct, along with the generation of carbon dioxide (CO₂) gas. A depolymerization conversion rate of the polycarbonate (PC) ranges from 90% to 99% (i.e., a conversion rate from high molecular weight compounds to small molecule chemicals, indicating that 90% to 99% of PC is depolymerized).

Finally, the bisphenol A (BPA) product can be recovered, for example, through a cooling crystallization process.

It is worth mentioning that the intermediate product diphenyl carbonate (DC) produced from the polycarbonate (PC) material can further depolymerize into phenol (R—OH) during the depolymerization reaction. Since phenol (R—OH) is the same compound as the single depolymerizing solvent used in the embodiment of the present disclosure, the weight ratio of the depolymerizing solvent (phenol) relative to the polycarbonate (PC) material will continuously increase during the depolymerization reaction, which is beneficial for the depolymerization reaction and the recovery of the depolymerizing solvent. Additionally, since the weight ratio of the depolymerizing solvent relative to the polycarbonate material will continuously increase during the depolymerization reaction, the initial amount of the depolymerizing solvent that is required can be reduced.

Furthermore, it is worth mentioning that through experiments, it has been found that the metal hydroxides (e.g., NaOH or KOH) at concentrations greater than 100 ppm, combined with the water content of 0.5 wt % to 10 wt %, facilitate the rapid depolymerization of polycarbonate (PC) to form bisphenol A (BPA) and carbon dioxide. Therefore, the polycarbonate depolymerization reaction can be carried out in a single depolymerizing solvent. If the water content is less than 0.5 wt %, the concentration of the metal hydroxide becomes too high (since the metal hydroxides dissociate in water to form hydroxide ions OH⁻ and metal cations Mn⁺), which may cause an undesirable reaction of the bisphenol A (BPA) product decomposing in the strong alkaline environment. Conversely, if the water content is greater than 10 wt %, the concentration of the metal hydroxide becomes too low, thereby reducing the overall depolymerization efficiency.

It is further worth noting that, in one embodiment of the present disclosure, 100 g of the polycarbonate (PC) material can be depolymerized into 89.7 g of BPA product and 17.3 g of CO₂ (gas), consuming about 7 g of water. Thus, during the depolymerization reaction, the water content in the reaction liquid will decrease. The method of the embodiment of the present disclosure controls the water content in the reaction liquid by monitoring and adding water, thereby achieving the technical effects described above.

In summary, to solve the technical problems existing in the related art, the present disclosure provides the method for degrading polycarbonate (i.e., transesterification technique for degrading polycarbonate), which includes using phenol as the single depolymerizing solvent. The polycarbonate material is subjected to the conditions where the metal hydroxide concentration is greater than 100 ppm (preferably 100 to 200,000 ppm, and more preferably 500 to 10,000 ppm) and, during the depolymerization reaction, the water content in the reaction liquid is controlled to be between 0.5 wt % and 10 wt %. As a result, the intermediate product (diphenyl carbonate) further depolymerizes into phenol (the same as the depolymerizing solvent) and carbon dioxide (which can be eliminated from the system exhaust or through CO₂ adsorption), thereby favoring the depolymerization reaction and increasing the polycarbonate depolymerization conversion rate up to 90% to 99%, with a bisphenol A (BPA) product yield of not less than 60%.

Moreover, the method for degrading polycarbonate according to the embodiment of the present disclosure effectively avoids the issue of cosolvent residues (e.g., toluene or dichloromethane) being left in the product (e.g., BPA) as mentioned in the related art.

### Experimental Data and Test Results

The content of the present disclosure is detailed in Exemplary Examples 1 to 3 and Comparative Example 1 described below. However, these examples are merely provided to aid in understanding of the disclosure and are not meant to limit the scope of the present disclosure.

Exemplary Example 1: 500 parts by weight of a depolymerizing solvent (i.e., phenol) is added into a reaction tank and heated to 80° C. (i.e., the first heating temperature). 4 parts by weight of a metal hydroxide aqueous solution (i.e., 32% NaOH aqueous solution) is added into the reaction tank and mixed with the depolymerizing solvent to form a mixed liquid. 200 parts by weight of polycarbonate (PC) particles and 35 parts by weight of water are added to the mixed liquid in the reaction tank to form a reaction liquid. The reaction liquid is heated to 120° C. (i.e., the second heating temperature) and continuously stirred for 5 hours to allow the polycarbonate (PC) particles to undergo depolymerization, thereby forming bisphenol A (BPA), phenol, and carbon dioxide. The concentration of the metal hydroxide (NaOH) in the reaction liquid is 1,732 ppm, and the water content in the reaction liquid is controlled to be about 4.7 wt %.

Exemplary Example 2: 500 parts by weight of a depolymerizing solvent (i.e., phenol) is added into a reaction tank and heated to 80° C. (i.e., the first heating temperature). 2 parts by weight of a metal hydroxide aqueous solution (i.e., 32% NaOH aqueous solution) is added into the reaction tank and mixed with the depolymerizing solvent to form a mixed liquid. 200 parts by weight of polycarbonate (PC) particles and 70 parts by weight of water are added to the mixed liquid in the reaction tank to form a reaction liquid. The reaction liquid is heated to 120° C. (i.e., the second heating temperature) and continuously stirred for 5 hours to allow the polycarbonate (PC) particles to undergo depolymerization, forming bisphenol A (BPA), phenol, and carbon dioxide. The concentration of the metal hydroxide (NaOH) in the reaction liquid is 829 ppm, and the water content in the reaction liquid is controlled to be about 9.0 wt %.

Exemplary Example 3: 500 parts by weight of a depolymerizing solvent (i.e., phenol) is added into a reaction tank and heated to 80° C. (i.e., the first heating temperature). 20 parts by weight of a metal hydroxide aqueous solution (i.e., 32% NaOH aqueous solution) is added into the reaction tank and mixed with the depolymerizing solvent to form a mixed liquid. 200 parts by weight of polycarbonate (PC) particles and 14 parts by weight of water are added to the mixed liquid in the reaction tank to form a reaction liquid. The reaction liquid is heated to 120° C. (i.e., the second heating temperature) and continuously stirred for 5 hours to allow the polycarbonate (PC) particles to undergo depolymerization, forming bisphenol A (BPA), phenol, and carbon dioxide. The concentration of the metal hydroxide (NaOH) in the reaction liquid is 8,719 ppm, and the water content in the reaction liquid is controlled to be about 1.4 wt %.

Comparative Example 1: 500 parts by weight of a depolymerizing solvent (i.e., phenol) is added into a reaction tank and heated to 80° C. (i.e., the first heating temperature). 4 parts by weight of a metal hydroxide aqueous solution (i.e., 32% NaOH aqueous solution) is added into the reaction tank and mixed with the depolymerizing solvent to form a mixed liquid. 200 parts by weight of polycarbonate (PC) particles are added to the mixed liquid in the reaction tank to form a reaction liquid. The reaction liquid is heated to 120° C. (i.e., the second heating temperature) and continuously stirred for 5 hours to allow the polycarbonate (PC) particles to undergo depolymerization. The concentration of the metal hydroxide (NaOH) in the reaction liquid is 1,818 ppm, and the water content in the reaction liquid is 0.18 wt %, which is uncontrolled.

Comparative Example 1 is prepared in a manner similar to Exemplary Example 1, except that no additional water is added into the reaction liquid of Comparative Example 1 during the depolymerization reaction, resulting in the water content being about 0.18 wt %, which is lower than the 4.7 wt % in Exemplary Example 1 and is below the 0.5 wt % requirement of the present disclosure.

Subsequently, Exemplary Examples 1 to 3 and Comparative Example 1 are tested to determine the depolymerization conversion rate of polycarbonate (PC) and the yield (%) of bisphenol A (BPA).

It is worth mentioning that, in the test results shown in Table 1, the depolymerization conversion rate of polycarbonate (PC) is determined as follows: first, 10 grams of crude reaction liquid (i.e., the reaction liquid after depolymerization) is taken. The 10 grams of crude reaction liquid is added to 50 grams of methanol and filtered to form a filter cake. After filtration, the filter cake is washed with another 50 grams of methanol. Then, the filter cake is dried, and the weight of the obtained solid(S) is recorded.

$$Formula of depolymerization conversion rate ( \% ) = ( 100 - S / ( 10 * ( PC / total weight of reaction liquid ) ) ) .$$

In this formula, S is the weight of the obtained solid after drying the filter cake, 10 grams is the weight of the crude reaction liquid taken out, PC is the initial amount of polycarbonate (PC) particles used, and the total weight of the reaction liquid is the total weight of the reaction liquid that contains water, depolymerizing solvent, metal hydroxide, and polycarbonate material.

Additionally, the product yield (%) of bisphenol A (BPA) is tested as follows: first, the crude reaction liquid after depolymerization is taken out and analyzed using high-performance liquid chromatography (HPLC) to determine the concentration A of BPA. Then, the product yield (%) of bisphenol A (BPA) is calculated using the following formula:

$$Product yield ( \% ) = A / ( PC / total weight of reaction liquid ) * 100.$$

In this formula, A is the concentration of BPA, PC is the initial amount of polycarbonate (PC) particles used, and the total weight of the reaction liquid is the total weight of the reaction liquid that contains water, depolymerizing solvent, metal hydroxide, and polycarbonate material.

TABLE 1
[Process Conditions and Test Results]

| | | Exemplary | Exemplary | Exemplary | Comparative |
|---|---|---|---|---|---|
| | Items | Example 1 | Example 2 | Example 3 | Example 1 |
| Process | Initial amount of | 500 | 500 | 500 | 500 |
| Conditions | depolymerizing solvent | | | | |
| | phenol | | | | |
| | (parts by weight) | | | | |
| | Initial amount of metal | 4 | 2 | 20 | 4 |
| | hydroxide aqueous | | | | |
| | solution (32% NaOH) | | | | |
| | (parts by weight) | | | | |
| | Initial amount of | 200 | 200 | 200 | 200 |
| | polycarbonate (PC) | | | | |
| | particles | | | | |
| | (parts by weight) | | | | |
| | Initial amount of water | 35 | 70 | 14 | — |
| | (H₂O) | | | | |
| | (parts by weight) | | | | |
| | Concentration of metal | 1,732 | 829 | 8,719 | 1,818 |
| | hydroxide in reaction | | | | |
| | liquid (ppm) | | | | |
| | Water content in | 4.7 | 9.0 | 1.4 | 0.18 |
| | reaction liquid (wt %) | | | | |
| Test | Depolymerization | 99 | 90 | 99 | 53 |
| Results | conversion rate of | | | | |
| | polycarbonate (PC) | | | | |
| | (%) | | | | |
| | Yield of bisphenol A | 89.3 | 73.2 | 63 | 38 |
| | (BPA) product (%) | | | | |

Table 1 shows that the process conditions in Exemplary Examples 1 to 3 meet the requirements of the present disclosure for the concentration of the metal hydroxide and the water content controlled in the reaction liquid, the depolymerization conversion rate of polycarbonate (PC) in each of Exemplary Examples 1 to 3 is not less than 90%, and the yield of bisphenol A (BPA) is not less than 60%.

Comparative Example 1 does not add additional water to the reaction liquid, and the water content in the reaction liquid is 0.18 wt % (uncontrolled), which is lower than the 4.7 wt % in Exemplary Example 1 and below the ideal requirement of 0.5 wt %. The depolymerization conversion rate of polycarbonate (PC) in Comparative Example 1 is 53%, which is significantly lower than the results of Exemplary Examples 1 to 3. Additionally, the yield of bisphenol A (BPA) in Comparative Example 1 is 38%, which is also significantly lower than the results of Exemplary Examples 1 to 3.

### Beneficial Effects of the Embodiments

In conclusion, in the method for degrading polycarbonate by the present disclosure, by virtue of “performing a preparation operation, which includes: providing a depolymerizing solvent, in which the depolymerizing solvent is phenol (phenol); performing an addition operation, which includes: adding a metal hydroxide to the depolymerizing solvent to form a mixed liquid; and performing a depolymerization operation, which includes: adding a polycarbonate (PC) material to the mixed liquid and adding a predetermined amount of water to the mixed liquid to form a reaction liquid,” and “an added concentration of the metal hydroxide being not less than 100 ppm, and a water content in the reaction liquid being controlled to be not greater 10 wt %,” the chemical reaction can incline towards a depolymerization reaction, thereby increasing the depolymerization conversion rate of polycarbonate and the yield of bisphenol A (BPA).

Further, the method for degrading polycarbonate provided by the present disclosure can effectively avoid the problem of cosolvent residues (e.g., toluene or dichloromethane) being left in the product (e.g., BPA) as mentioned in the related art.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for degrading polycarbonate, comprising:
performing a preparation operation, which includes providing a depolymerizing solvent, wherein the depolymerizing solvent is phenol;
performing an addition operation, which includes adding a metal hydroxide to the depolymerizing solvent to form a mixed liquid; and
performing a depolymerization operation, which includes adding a polycarbonate material to the mixed liquid, and adding a predetermined amount of water to the mixed liquid to form a reaction liquid;
wherein an added concentration of the metal hydroxide is not less than 100 ppm, and a water content in the reaction liquid is controlled to be not greater than 10 wt %; wherein the polycarbonate material undergoes a depolymerization reaction in the depolymerization operation to form bisphenol A (BPA) and carbon dioxide (CO₂).

2. The method for degrading polycarbonate according to claim 1, wherein the preparation operation further includes heating the depolymerizing solvent to a first heating temperature ranging from 60° C. to 100° C.; wherein the depolymerization operation further includes heating the reaction liquid to a second heating temperature ranging from 110° C. to 150° C.

3. The method for degrading polycarbonate according to claim 2, wherein the first heating temperature ranges from 70° C. to 90° C., and the second heating temperature ranges from 110° C. to 140° C.

4. The method for degrading polycarbonate according to claim 1, wherein the depolymerization operation further includes controlling the water content in the reaction liquid to be between 0.5 wt % and 10 wt % during the depolymerization reaction.

5. The method for degrading polycarbonate according to claim 4, wherein, when the water content in the reaction liquid is consumed to be below 0.5 wt %, the depolymerization operation further includes replenishing water to the reaction liquid to adjust the water content in the reaction liquid to be between 0.5 wt % and 10 wt %.

6. The method for degrading polycarbonate according to claim 1, wherein an initial weight ratio of the depolymerizing solvent relative to the polycarbonate material in the reaction liquid ranges between 1 to 14.

7. The method for degrading polycarbonate according to claim 1, wherein an intermediate product formed by the depolymerization of the polycarbonate material is diphenyl carbonate, which is further depolymerized into phenol and carbon dioxide (CO₂) during the depolymerization reaction; and wherein a weight ratio of the depolymerizing solvent relative to the polycarbonate material continuously increases during the depolymerization reaction.

8. The method for degrading polycarbonate according to claim 1, wherein the metal hydroxide is at least one material selected from the group consisting of alkali metal (Group 1A metal) hydroxides, alkaline earth metal (Group 2A metal) hydroxides, and transition metal hydroxides.

9. The method for degrading polycarbonate according to claim 8, wherein the metal hydroxide is at least one of sodium hydroxide (NaOH) and potassium hydroxide (KOH).

10. The method for degrading polycarbonate according to claim 1, wherein the added concentration of the metal hydroxide ranges from 500 ppm to 10,000 ppm.

11. The method for degrading polycarbonate according to claim 1, wherein the addition operation includes adding an aqueous solution containing the metal hydroxide to the depolymerizing solvent to form the mixed liquid.

12. The method for degrading polycarbonate according to claim 11, wherein a weight percentage concentration of the metal hydroxide in the aqueous solution ranges from 15 wt % to 60 wt %.
